(19) United States
(12) Patent Application Publication
(10) Pub. No.: US 2008/0298548 A1
(43) Pub. Date: Dec. 4, 2008

(54) Device for X-Ray Brachytherapy, and Method for Positioning a Probe Introduced Into a Body for X-Ray Brachytherapy

(76) Inventors: Jens Fehre, Hausen (DE); Bernd Granz, Oberasbach (DE); Markus Lanski, Wasseralfingen (DE); Ralf Nanke, Neunkirchen am Brand (DE)

Correspondence Address: SCHIFF HARDIN, LLP;PATENT DEPARTMENT, 6600 SEARS TOWER, CHICAGO, IL, 60606-6473, US

(21) Appl. No.: 12/094,925

(22) PCT Filed: Sep. 28, 2006
(86) PCT No.: PCT/EP2006/066834

§ 371 Date: May 23, 2008

(30) Foreign Application Priority Data
Nov. 24, 2005 (DE) 10 2005 056 066.0

(51) Int. Cl. A61N 5/10

(52) U.S. Cl. 378/65

(57) ABSTRACT

In a method and device for x-ray brachytherapy, a probe is inserted into and moved within a living subject, the probe having a distal end, an x-ray source carried by said probe at said distal end that irradiates an x-ray beam into an exposure area outside of the probe, a mark generator carried by said probe at said distal end that generates a mark that identifies at least a portion of said exposure area, and an optical observation system, having an optical detector carried at said distal of said probe, that generates an optical image of said at least one part of said exposure area that is identified by said marking, the marking being visible in said optical image.

### BACKGROUND OF THE INVENTION

1. Field of the Invention

The invention concerns a device for x-ray brachytherapy as well as a method for positioning of a probe inserted into the inside of a body for x-ray brachytherapy.

2. Description of the Prior Art

X-ray brachytherapy is a therapeutic treatment with x-rays in which the x-ray source is brought very close to the tissue to be treated (for example a tumor or a vessel wall) after the implementation of a endovascular dilatation. In order to able to insert the x-ray source with the aid of a catheter or a probe either without an invasive procedure or with an optimally minimally-invasive procedure inside a body, a miniaturized x-ray source is required as is known from U.S. Pat. No. 6,721,392, for example. This is arranged at the distal end of a probe that, for example, is intraoperatively positioned in a tumor or tumor bed (after its extraction), as is explained in detail in the PR information of Carl Zeiss AG, Medical Engineering Innovation by Carl Zeiss AG, “Intraoperative Strahlentherapie mit dem INTRABEAM System von der Carl Zeiss AG,” September 2004, for example.

A miniaturized x-ray source that is arranged in a catheter with which it can be inserted into the body cavities (lumen) in order to irradiate selected tissue zones from the immediate surroundings from there is known from United States Patent Application Publication 2003/0149327 A1. It contains a shielding rotatable around the axis of the catheter in order to radiate the x-rays in a targeted manner at least perpendicular to the axis in a selected solid angle. The surroundings of the catheter can be observed with an optical observation device arranged in a catheter. A light source that exposes only the part of the surface of the hollow space that is also irradiated is used for this purpose.

In endovascular brachytherapy with a beta or gamma radiator arranged in the tip of a catheter, it is also known from DE 10 2004 008 373 B3 (for example) to arrange an optical observation device in the catheter. For this purpose a brachytherapy catheter is integrated into a unit with an OCT catheter operating on the basis of optical coherence tomography (OCT).

For the therapeutic success it is essential that the x-rays radiating out of the catheter from the x-ray source in an exposure area for most part exclusively strike the tissue (for example the tumor) to be treated in order to ensure an optimally low exposure of the healthy tissue located near this. This requires a precise positioning of the exposure area, i.e. a precise positioning and alignment of the x-ray source or of the solid angle in which the x-rays exit.

### SUMMARY OF THE INVENTION

An object of the present invention is to provide a device for x-ray brachytherapy with a probe that can be inserted inside a body, with which probe a precise positioning of the exposure area is possible. A further object of the invention is to provide a method for positioning a probe inserted inside a body for x-ray brachytherapy.

With regard to the device, the cited object is achieved by a device containing a probe that can be inserted into the inside of a body, the probe at its distal end having an x-ray source that radiates an x-ray beam into an exposure area outside of the probe, as well as an optical observation device for generation of an optical image rendering at least one part of the exposure area that is identified by a marking in the optical image. This enables a precise positioning of the exposure area, i.e. a precise positioning of the x-ray source and of the solid angle range in which the x-rays generated by the x-ray source are radiated.

As used herein the term “probe” is an instrument that can be introduced into the inside of a body. This can be both a catheter (in the narrower sense) that is inserted into body cavities (transluminal) and a needle-like instrument that can be placed within a tissue zone (percutaneous or interstitial).

When the optical observation device comprises an imaging device operating according to the OCT method, tissue structures are particularly clearly rendered in the field of view of the observation device.

In a preferred embodiment of the method, at least one light source associated with the x-ray source is arranged in the probe, which light source emits light that intracorporeally marks the radiation area. In a preferred embodiment, a number of light sources are provided for this that respectively emit an approximately parallel ray beam that propagates at least approximately along the boundary rays of the x-ray beam. The active region of x-ray radiation striking the surface of the tissue zone to be treated can be rendered particularly precisely in this manner and independent of the position and the shape of the surface of the tissue zone to be treated.

If the optical axis of the observation device in the subject space coincides with the center axis of the x-ray beam, it is ensured that the image center simultaneously renders the position of the center axis of the x-ray beam. The active region of the x-rays can then be mixed into the image, for example as a circular line given a conical x-ray beam.

If the exposure area can be adjusted relative to the probe, a particularly high therapeutic flexibility is ensured. Moreover, if a software for image evaluation as well as for automatic (possibly successive) positioning of the exposure area is provided in a control and evaluation device such that a treatment area previously marked in the optical image is exposed with predeterminable x-ray parameters, operator errors during the therapeutic treatment are largely avoided.

In a particularly advantageous embodiment of the device, a software for automatic localization and marking of the treatment area is implemented in the control and evaluation device.

A device according to the invention is in particular suitable for insertion into a urethra or a ureter and for therapeutic treatment of a prostate, bladder or kidney tumor.

The above object also is achieved in accordance with the present invention by a method for positioning a probe for x-ray brachytherapy, including the steps of introducing a probe into the interior of a living body that carries, at its distal end, and x-ray source, radiating x-rays from the x-ray source into an exposure area within the body outside of the probe, identifying at least a portion of the exposure area with a marking, and generating an optical image that shows at least the aforementioned portion of the exposure area with an optical observation device, with the aforementioned portion being identified by the marking in the optical image.

The aforementioned advantages associated with the device according to the invention are also achieved by the method according to the invention.

If a marker or a luminophore is applied that preferably accumulates in a tumor and that is made visible in the optical image via excitation with electromagnetic radiation, the exposure area can be correctly positioned in a particularly simple and illustrative manner in that the exposure area is brought into congruence with the area marked by the luminophore.

If an x-ray-sensitive dye is applied that accumulates in a tumor and changes color upon exposure with x-rays, which areas of the tumor have already been treated is visible.

A method according to the invention is in particular suitable in the treatment of a prostate, bladder or kidney tumor in which the probe is inserted into a urethra or a ureter.

### BRIEF DESCRIPTION OF THE DRAWINGS

FIG. 1 schematically illustrates an embodiment of a device for x-ray brachytherapy constructed and operating in accordance with the present invention.

FIGS. 2, 3 and 4 respectively illustrate optical images obtained with the device shown in FIG. 1, in which the exposure area is identified by a marking that is visible in the optical image.

### DESCRIPTION OF THE PREFERRED EMBODIMENTS

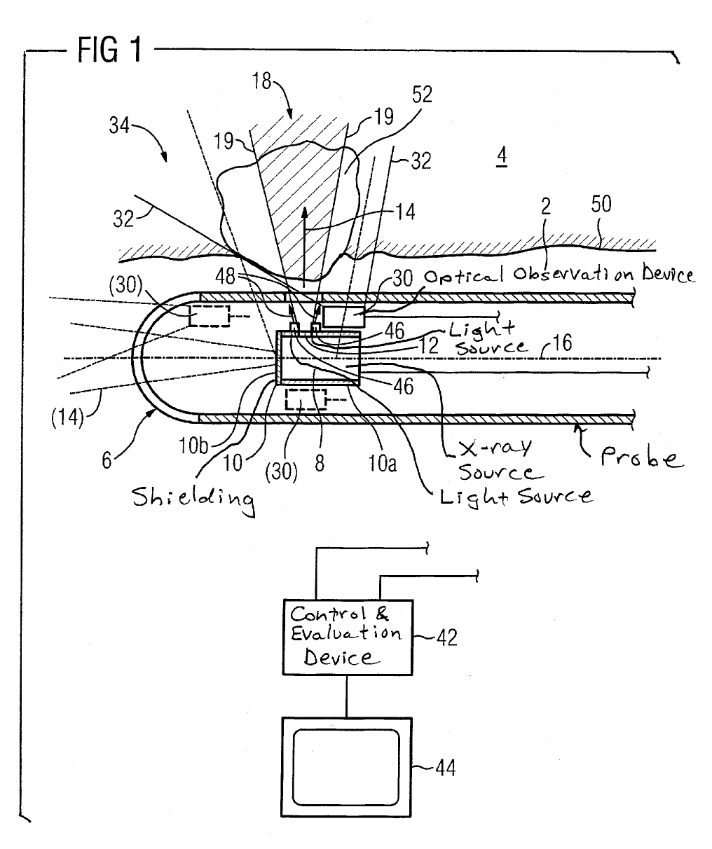

According to FIG. 1, a probe 6 (a catheter in the example) in which an x-ray source 8 is arranged at its distal end is inserted into a cavity (lumen) 2 of a body 4 (which can be a urethra or a ureter). A shielding 10 is associated with the x-ray source 8, which shielding 10 in the exemplary embodiment contains a cylindrical part 10a that is provided in its circumference with a diaphragm or aperture 12 through which an x-ray beam 14 can exit perpendicular to the longitudinal axis 16 of the probe 6, i.e. radially in a (for example conical) exposure area 18 defined by the shape of the aperture 12 and its distance from the anode of the x-ray source 8, which exposure area 18 is emphasized by hatching in FIG. 1 and is indicated by boundary rays 19.

The cylindrical part 10a of the shielding 10 is arranged within the probe 6 such that it can be rotated around its longitudinal axis 16 so that the exposure area 18 can likewise be pivoted on this longitudinal axis 16.

The shielding 10 has on its front side a front plate 10b that is provided with a closable diaphragm (not shown in detail in FIG. 1) with which it is possible to selectively radiate an x-ray beam 14 in the direction of the longitudinal axis 16. In this case either a movable lock is provided with which the aperture 12 can be closed or an additional shielding is provided that is arranged in the probe 6 such that the aperture 12 can be positioned in the region of this shielding.

An optical observation device 30 with a field of view 34 illustrated by boundary lines 32 (in particular an optical observation device 30 operating according to the OCT method) is arranged in the immediate proximity of the x-ray source 8 in the region of the aperture 12, with which optical observation device 30 a subject region can be observed that renders (shows) at least a portion of the exposure area 18.

The probe 6 is connected to a control and evaluation device 42 with which the x-ray source 8 and the observation device 30 are controlled and the signals transmitted from the observation device 30 are evaluated so that they can be shown as an optical image on a display device 44 (for example a monitor).

In the example of FIG. 1 a tissue zone 52 to be therapeutically treated (for example a tumor, in particular a prostate tumor, a bladder tumor or a kidney tumor that should be irradiated with x-rays 14) is located in the region of the wall 50 of the cavity 2.

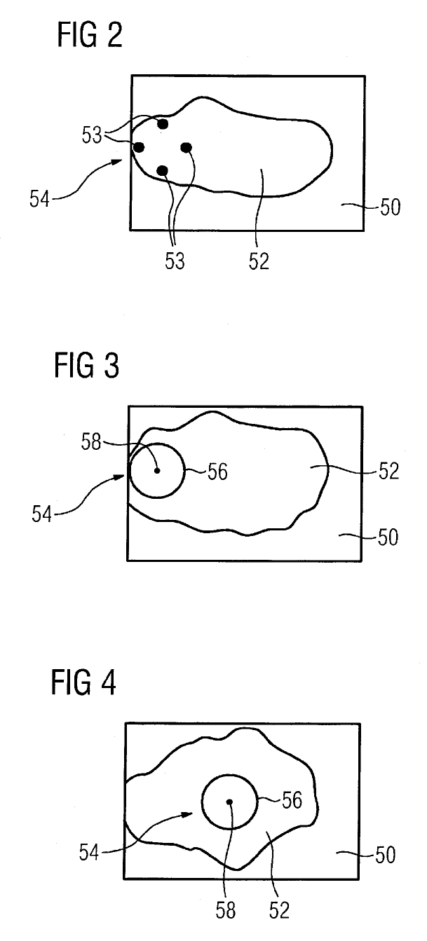

According to FIG. 2, the wall 50 of the cavity with the tissue zone 52 is visible in the optical image of the surroundings if the probe given corresponding illumination. Moreover, four points of light are detectable in the image as they are generated by four light sources 46, for example, and that show a direct marking 54 of the intersection surface of the exposure area 18 with the wall 50 of the cavity 2.

If a luminophore or marker is additionally applied (for example directly with the aid of the probe) that preferably accumulates in the tissue zone 52 if it is a tumor and that is excited by electromagnetic radiation (for example by the light source used for illumination) and emits fluorescence light in the visible range, the detectability of the tumor is clearly improved at least in its surface area with which it borders the cavity, such that it can also be automatically localized and bounded with an image recognition software. A largely automated positioning of the probe and therapeutic treatment of the tumor with the aid of x-ray parameters (dose rating, anode current, tube voltage and exposure duration) that have been predetermined by a therapist using the present finding is possible in this manner given a likewise known position of the exposure area that, in the example, can likewise be identified by the image detection software using the points of light 53.

As an alternative to this, the treatment area can also be manually marked by the therapist. In this case as well as the therapeutic treatment can also be largely automated in that, given a resting probe, the treatment area is possibly automatically positioned multiple times after an occurred manual marking so that the treatment area is entirely detected.

As an alternative to the direct marking according to FIG. 2, the marking 54 can be electronically mixed into the image with an image processing software implemented in the control and evaluation device. According to FIG. 3, the envelope of the x-ray brachytherapy striking the surface of the wall 50 can then be mixed into as a circular line 56 and its center axis as a point 58.

In FIG. 4 an exemplary embodiment is illustrated in which the center axis of the field of view of the observation device 30 coincides with the center axis of the x-ray beam. In this case a precise positioning is simplified since errors that can be caused by oblique and offset center axes are avoided.

The invention is presented using a catheter inserted into the cavity of a body. In principle the invention is also suitable for probes that are directly inserted into the tissue, as this is the case in the invasive post-treatment (explained above) of a tumor bed of a previously removed tumor. The tissue zone to be treated can also be a vessel wall that should be irradiated after the implementation of a dilatation to reduce the restenosis rate.

Although modifications and changes may be suggested by those skilled in the art, it is the intention of the inventors to embody within the patent warranted heron all changes and modifications as reasonably and properly come within the scope of their contribution to the art.

We claim as our invention:

1-21. (canceled)

22. A device for x-ray brachytherapy comprising:
a probe configured for insertion into and movement within a living subject, said probe having a distal end;
an x-ray source carried by said probe at said distal end that radiates an x-ray beam into an exposure area outside of the probe;
a mark generator carried by said probe at said distal end that generates a mark that identifies at least a portion of said exposure area; and
an optical observation system, comprising an optical detector carried at said distal end of said probe, that generates an optical image of said at least one part of said exposure area that is identified by said marking, said marking being visible in said optical image.

23. A device as claimed in claim 22 wherein said optical observation system is OCT system.

24. A device as claimed in claim 22 wherein said mark generator comprises at least one light source associated with said x-ray source that emits light that intracorporeally marks said exposure area, as said marking.

25. A device as claimed in claim 24 wherein said mark generator comprises a plurality of light sources that respectively emit substantially parallel light beams that propagate at least approximately along boundary rays of said x-ray beam.

26. A device as claimed in claim 22 wherein said optical detector has an optical axis that coincides with a center axis of said x-ray beam.

27. A device as claimed in claim 22 wherein said x-ray source is operable to adjust a position of said exposure area relative to said probe.

28. A device as claimed in claim 27 comprising a control device supplied with said image from said optical observation system, said control device being configured to automatically evaluate said optical image to identify said marking therein and to control adjustment of the position of said exposure area relative to said probe dependent on the identification of the marking in the optical image.

29. A device as claimed in claim 28 wherein said control device is configured by programming for automatic localization and marking of a treatment area in the living body to be radiated with said x-ray beam for brachytherapy.

30. A method for x-ray brachytherapy comprising:
inserting a probe into a living subject, said probe having a distal end;
from an x-ray source carried by said probe at said distal end, irradiating an x-ray beam into an exposure area outside of the probe to implement brachytherapy in said exposure area;
with a mark generator carried by said probe at said distal end, generating a mark that identifies at least a portion of said exposure area; and
with an optical detector carried at said distal end of said probe, generating an optical image of said at least one part of said exposure area that is identified by said marking, said marking being visible in said optical image.

31. A method as claimed in claim 30 comprising employing an OCT system as said optical observation system.

32. A method as claimed in claim 30 comprising, with said mark generator, emitting light that intracorporeally marks said exposure area, as said marking.

33. A method as claimed in claim 30 comprising, with said mark generator, emitting a plurality of substantially parallel light beams that propagate at least approximately along boundary rays of said x-ray beam.

34. A method as claimed in claim 30 comprising orienting said optical detector so that an optical axis thereof coincides with a center axis of said x-ray beam.

35. A method as claimed in claim 30 comprising, from within said probe, operating said x-ray source to adjust a position of said exposure area relative to said probe.

36. A method as claimed in claim 35 comprising automatically evaluating said optical image to identify said marking therein and controlling adjustment of the position of said exposure area relative to said probe dependent on the identification of the marking in the optical image.

37. A method as claimed in claim 36 comprising automatically localizing and marking a treatment area in the living body to be radiated with said x-ray beam for brachytherapy.

38. A method as claimed in claim 30 comprising inserting said probe into a ureter or urethra of said subject.

39. A method as claimed in claim 30 comprising situating said probe in said subject to irradiate tumerous tissue in said exposure area, selected from the group consisting of tumerous prostate tissue, tumerous bladder tissue, and tumerous kidney tissue.
